(19) United States
(12) Patent Application Publication
(10) Pub. No.: US 2021/0170122 A1
(43) Pub. Date: Jun. 10, 2021

(54) METHOD OF WEIGHT REGULATION USING NEBULIZED NICOTINE

(71) Applicant: BN Intellectual Properties, Inc., St. Petersburg, FL (US)

(72) Inventors: Nicholas A. HAVERCROFT, Scunthorpe (GB); Donald M. PELL, St. Petersburg, FL (US)

(21) Appl. No.: 17/174,107

(22) Filed: Feb. 11, 2021

Related U.S. Application Data
(63) Continuation-in-part of application No. 16/025,586, filed on Jul. 2, 2018, now abandoned.
Application No. 16/025,586 is a continuation-in-part of application No. 15/234,035, filed on Aug. 11, 2016, now abandoned.

(51) Int. Cl. A61M 15/06; A24F 40/05; A24F 40/10; A24F 40/50; A61K 31/465; A61K 9/00

(52) U.S. Cl. CPC A61M 15/06; A24F 40/05; A24F 40/10; A61M 15/0085; A61K 31/465; A61K 9/0078; A24F 40/50

(57) ABSTRACT

A method of regulating weight in a patient includes operations of measuring a patient weight, determining a dose of nicotine which does not trigger nicotine intolerance, delivering the dose of nicotine to the patient by inhalation from an active mesh nebulizer at a repeated interval, measuring the weight of the patient at a weight monitoring interval, and determining, based on the patient weight and patient behavior, whether to adjust the nicotine dose.

### PRIORITY CLAIM

The present application claims priority to the following previously-filed patent applications, and incorporates the contents thereof herein in their entirety: U.S. patent application Ser. No. 16/025,586, entitled MEDICAL PRODUCT AND METHOD FOR ELIMINATING SYMPTOMS OF NICOTINE WITHDRAWAL, filed on Jul. 2, 2018.

The present application also relates to the subject matter of the following patent applications and incorporates the contents thereof herein in their entirety: U.S. patent application Ser. No. 16/547,478 NICOTINE FORMULATION FOR ACTIVE MESH NEBULIZER filed on Aug. 21, 2019, U.S. patent application Ser. No. 16/547,072 METHOD OF DELIVERING PHARMACEUTICAL PRODUCTS, filed on Aug. 21, 2019, and U.S. patent application Ser. No. 16/836,485 NEBULIZER WITH CONTROLLED DOSAGE, filed on Mar. 31, 2020.

### BACKGROUND

Smoking cigarettes and the use of other tobacco products is associated with many adverse health conditions, such as lung cancer, emphysema, and heart disease. Smoking cessation to avoid the adverse health conditions associated with smoking cigarettes and use of other tobacco products is believed to be beneficial to long term individual and public health. Smoking cessation is frequently associated with weight gain.

### BRIEF DESCRIPTION OF THE FIGURES

FIG. 1 is a flow diagram of a method of delivering nicotine for regulating weight, in accordance with some embodiments.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. The present disclosure may repeat reference numerals and/or letters in the various examples of operations. This repetition is for the purpose of simplicity and clarity and does not in itself dictate a relationship between the various embodiments and/or configurations discussed.

Nicotine produces a brain effect and metabolic effect, which increases or enhances metabolism in the nicotine user, causing increased calorie consumption and heightened metabolic rate. In some persons, nicotine also promotes appetite suppression. Thus, many persons take up smoking in order to lose weight, or to regulate weight.

Many persons who quit smoking experience weight gain during the course of smoking cessation. Unexpected or undesirable weight gain during smoking cessation is one reason that some persons return to smoking tobacco products, anticipating that continuing tobacco use (e.g., nicotine consumption) will result in weight loss. Reducing the amount of tobacco use for individual patients, and for large populations, has been shown to effectively decrease the frequency of these and other illnesses associated with tobacco consumption.

The use of tobacco to ingest nicotine is associated with many negative health conditions. The use of nicotine-containing solutions to promote weight regulation is believed to avoid the negative health conditions associated with tobacco use.

The present disclosure relates to a method of delivering nicotine to a patient for weight regulation. For some persons, performing the methods described hereinbelow is associated with maintaining weight during smoking cessation. For some persons, performing the methods described hereinbelow is associated with reducing weight, regardless of the person's smoking history.

An active mesh nebulizer is uniquely effective at generating a stream of droplets or particles of a liquid contained therein into the lungs of a patient upon inhalation of the generated stream of droplets or particles. The liquid in an active mesh nebulizer is converted into droplet form upon vibration of a piezoelectric active mesh, which vibrates at between 50 and 400 kHz, to force the liquid through small openings in the piezoelectric active mesh with each vibration. Droplets of liquid which are formed after the liquid is forced through the piezoelectric active mesh are directed into a mouthpiece for inhalation by a person using the active mesh nebulizer. The active mesh nebulizer forms droplets of the liquid therein without heating the liquid, which preserves chemical compounds dissolved in the liquid for delivery to a user upon inhalation. Droplets having a particle diameter of between 0.5 and 5.0 μm are entrained with inhaled air deep into the lungs, and absorbed through the alveoli into the bloodstream.

A unique and unexpected feature of delivering chemical compounds to a user through the lungs by inhaling droplets with diameters of between 0.5 and 5.0 μm from an active mesh nebulizer is that chemical compounds which are so delivered have an unexpectedly large effect on the brain, despite the small total quantity of chemical compound which is so delivered. Particles having a diameter greater than about 5 microns have sufficient mass that, upon inhalation, the particles strike the walls of the mouth, throat, and upper branches of the lungs, where absorption rates are low, as compared to the absorption rates of inhaled droplets in the lower branches of the lungs and the alveolar sacs.

Venous blood traveling through the lungs to be oxygenated absorbs the chemical compounds delivered to the lungs, returns to the heart, and is then circulated throughout the body. Approximately 20% of the blood which exits the heart is directed to the brain, where the chemical compound absorbed therein is rapidly perfused into the brain tissue via the large surface area of the complex capillary network in the brain. Thus, a small concentration of a brain active chemical compound is able to achieve saturation, or near saturation, of the chemical receptors for that compound in the brain tissue, and trigger the anticipated brain effect of that brain active chemical compound.

For active mesh nebulizers which deliver droplets of nicotine solution, through the lungs, to the brain, the brain effect experienced by a person using the active mesh nebulizer is similar to the brain effect experienced by smoking tobacco or using other nicotine-containing products such as electronic cigarettes, but the harmful side effects of tobacco consumption are avoided. Further, because of the efficiency of generating a brain effect with a brain-active chemical compound by the method of delivery described above, the concentrations of the brain-active chemical compound in the nebulizer solution (e.g., the nicotine solution), and the total dose of delivered medication to achieve the brain effect, are smaller than expected. Further description of treatment of a patient with an active mesh nebulizer using a nicotine-containing solution are provided below. Nicotine delivered by active mesh nebulizer is effective at producing the brain effect, whether delivered as the free base in solution, or as a dissolved nicotine salt. Because the droplets of solution with diameters of between 0.5 and 5.0 μm are entrained so efficiently into the deep lung, or alveolar tissue, there is no need to modify the pH of the solution to avoid triggering the cough reflex in a person using the active mesh nebulizer. Thus, a nebulizer solution has, in some embodiments, a pH ranging from about 3.5 to about 12 with no negative impact on the lungs or nicotine absorption when delivered from an active mesh nebulizer.

Nicotine has a flavor that many nicotine users perceive to be strong and bitter. In some embodiments, a flavoring is added to the mixture to mask or moderate the strong or bitter flavor of nicotine. In some embodiments, the nicotine solution (nebulizer solution) further comprises a water soluble flavoring component added to modify the flavor of the solution after delivery of the compound to a user from an active mesh nebulizer. The small diameter of the particles or droplets produced by an active mesh nebulizer have a large overall surface area which promotes evaporation of the droplet and/or of components in the droplet. The evaporation of flavoring compounds enhances the flavor of the components in the nicotine solution. In some embodiments, the flavoring component added to the nicotine solution is a tobacco flavoring extract. In some embodiments, the flavoring component added to the nicotine solution includes menthol. In some embodiments, other flavoring components are added to the nicotine solution, including fruit flavorings, and/or the like.

Thus, nicotine substitution is an effective part of a weight regulation plan for a person seeking to [1] avoid weight gain upon smoking cessation, or [2] induce weight loss upon substitution of nicotine (nebulized) for tobacco product consumption, or addition of nicotine for a person seeking a metabolic change to promote initial weight loss in a weight regulation plan. For some persons, nicotine substitution, or nicotine addition, is part of a weight regulation plan which includes exercise and dietary changes.

In some embodiments, the nicotine treatment program (nicotine substitution, or nicotine addition) is performed for a short period of time, until the metabolic rate change to promote weight loss has reduced the patient weight, and nicotine treatment is tapered or halted entirely as changes to diet and activity levels supplement the metabolic change induced by the delivered nicotine of a first part of the weight treatment program. In some embodiments, the nicotine treatment program comprises solely of substitution of nicotine from an active mesh nebulizer for nicotine from tobacco consumption, and the person so treated maintains a steady rate of nicotine substitution. Persons of ordinary skill, being instructed in the effectiveness of nicotine treatment programs comprising using an active mesh nebulizer to substitute or add nicotine in a person's metabolism as described above, will understand that numerous other variations of diet, activity change, and nicotine delivery patterns are also included within the scope of the present disclosure.

The nicotine treatment with a nicotine solution from an active mesh nebulizer, as part of a weight regulation plan, avoids the adverse health effects of tobacco consumption and electronic cigarette use. Further, a patient is more likely to achieve a weight regulation effect (i.e., maintain low weight or lose weight) with an active mesh nebulized nicotine solution than with the smoking cessation products described above because smoking cessation products, such as transdermal patches or nicotine gum, have a low frequency of patient adherence. Patients quit transdermal patches or nicotine gum because these smoking cessation products do not have at least the same brain effect as smoking tobacco products. Active mesh nebulized nicotine solution produces a brain response similar to the effect of smoking tobacco products, and has a higher likelihood of patient adherence to the nicotine treatment/weight management plan.

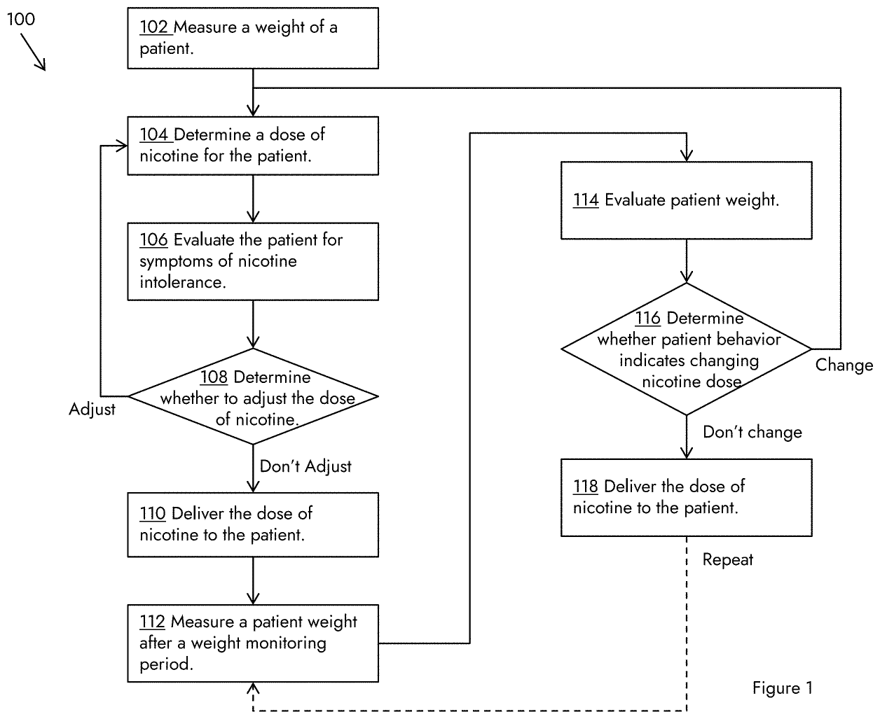

FIG. 1 is a flow diagram of a method 100 for delivering nicotine to a patient, in accordance with some embodiments. In operation 102 of method 100, a patient is weighed to determine a first weight of a weight management plan. In some embodiments, the patient is weighed at a physician's office in order to perform an initial patient evaluation as part of creating the weight management plan. In some embodiments, for patients with a history of tobacco use, or smoking, the initial weight serves as a baseline for weight maintenance during smoking cessation. In some embodiments, for patients with little or no history of using tobacco products, the initial weight is used as a basis for monitoring weight loss during smoking cessation (nicotine substitution/nicotine addition to a patient metabolism). In some embodiments, the weight management plan relies solely on the treatment by inhaled nicotine solution as described below. In some embodiments, the weight management plan includes a combination of nicotine treatment as described below in Method 100, in conjunction with diet change and/or changes to patient physical activity levels (e.g., a program of physical therapy and/or exercise).

Due diligence is exercised in performing operations 104, 106, and 108, as described below, to monitor patient health, and to avoid patient discomfort, harm, and/or nicotine overdose. For purposes of the present disclosure, the terms “first dose,” “second dose,” and so forth, indicate discrete quantities of nicotine to be delivered to a patient at a regular interval or a repeated interval in a plume of droplets of nicotine solution. The term “initial dose” refers to a quantity of nicotine which is used for an evaluation period to determine whether a patient exhibits nicotine intolerance. In some embodiments, the initial dose of nicotine is at least one incremental dose of nicotine delivered until the patient exhibits symptoms of nicotine intolerance. A cumulative dose of nicotine is a dose of nicotine delivered incrementally (e.g., in at least one incremental dose) to a patient during an evaluation period to determine nicotine intolerance. The first dose of nicotine is either the same as a single incremental dose of nicotine used in the nicotine intolerance evaluation process, or a cumulative dose (e.g., multiple initial doses) delivered during nicotine intolerance evaluation.

The first dose of nicotine is delivered to a patient at a repeated interval (e.g., for several days, or weeks, or months), until a patient has undergone some weight loss due to a change in the patient's metabolic rate as a result of the first dose of nicotine, or it is determined that the first dose should be replaced by a second dose to trigger the metabolic change and correlating weight loss. In some embodiments, the change in nicotine dosage is an increase in nicotine dosage. In some embodiments, the change in nicotine dosage is a decrease in nicotine dosage.

In operation 104 of method 100, a dose of nicotine for the patient is determined. The dose of nicotine delivered to a patient is determined as a function of the patient's physiology (age, weight, biological sex), previous smoking history, nicotine tolerance, and so forth. Nicotine doses are delivered by nebulizing a nicotine source solution containing nicotine and water, as described herein. In some embodiments, other ingredients are also included in the nebulized source solution (flavorings, and so forth) in order to promote adherence to the weight regulation plan by making the nicotine treatment more pleasant for the patient.

For purposes of clarity, biological males are referred to hereinafter as men, and biological females are referred to hereinafter as women. Because men and women have different metabolic responses to nicotine consumption, some weight regulation plans factor a person's biological gender into the nicotine treatment dose sizes. In some embodiments, an initial dose delivered to a patient for nebulization is the same regardless of biological sex. In general, men experience larger weight fluctuations (both weight gain and weight loss) than women experience when starting or stopping nicotine use. Weight fluctuations associated with nicotine use are believed to be caused by metabolic stimulation and/or appetite suppression. For example, in a weight loss scenario, metabolic increase and/or appetite suppression often results in weight loss punctuated by periodic periods of a weight “plateau” where weight change almost stops. Adjusting a nicotine dose, or waiting for a period of about 2-4 weeks, is typically sufficient to see weight loss continue after a weight plateau period.

In some embodiments, a person whose previous smoking history indicates a high tolerance for nicotine (e.g., the person was a heavy smoker) is prescribed a large dose of nebulized source solution, and a person whose previous smoking history indicates a low tolerance for nicotine (e.g., the person was a light smoker, or a non-smoker) is prescribed a small dose of nebulized source solution.

U.S. patent application Ser. No. 16/547,072 METHOD OF DELIVERING PHARMACEUTICAL PRODUCTS, filed on Aug. 21, 2019, incorporated herein by reference, describes a process of measuring and delivering a dose of a pharmaceutical product to a patient. For purposes of the present disclosure, a dose of nicotine delivered to a patient as part of a nicotine treatment in a weight regulation plan comprises a single quantity of nicotine delivered to a patient in a short period of time in one, or several sequential or near-sequential, inhalations, followed by a longer period of time (typically hours, or hundreds or thousands of inhalations) between doses.

A dose of nicotine is generated by activating the active mesh nebulizer as described below. An active mesh nebulizer is a medical device configured to deliver the liquid formulation to a patient as a plume of small particles inhaled into the lungs. In some embodiments, the active mesh nebulizer is configured to deliver the plume of droplets or particles of liquid to a user for treatment of lung tissue, or absorption into the body through the lungs for treatment of other tissues in a patient. An active mesh nebulizer is configured to receive and nebulize liquid formulations from a vial or capsule containing the liquid formulation which includes a chemical compound. In some embodiments, the liquid formulation in the vial comprises multiple doses of medication. The vial is configured to be replaced with a new vial from time to time.

The active mesh nebulizer dispenses liquid formulations of medication from the vial as an aerosol plume, or a plume of droplets or particles of liquid, according to the digitally stored instructions performed by the active mesh nebulizer.

The instructions for the active mesh nebulizer relate to parameters associated with operating the active mesh nebulizer to deliver a plume of particles to a patient or user, to determine the total dose of the medication in the liquid formulation for a patient or user's care, and/or the timing associated with operating the active mesh to deliver medications to the patient or user. An active mesh nebulizer has an active mesh, a sheet of material (metal or a polymer) having holes therein and connected to a piezoelectric element. During plume generation, the active mesh is in direct contact with the liquid formulation while a controller starts and stops vibration of the active mesh, or starts and stops the piezoelectric element connected to the active mesh. Plume generation occurs during mesh vibration, resulting in liquid being forced through the holes in the active mesh and into a volume of air (for example within a mouthpiece) where the particles are positioned for inhalation. By directing the plume of particles toward a patient mouth (e.g., by directing the particles into the mouthpiece volume prior to or during inhalation), the generated particles are absorbed into the lungs during inhalation, with no waste. By ending the plume generation prior to the end of an inhalation, the entirety of the plume is absorbed by the patient through the lungs and no liquid, or chemical compound in the liquid, is wasted. In some embodiments, the active mesh vibration starts after a patient or user inhales, and stops before a patient or user stops the inhalation. In some embodiments, the instructions for the active mesh nebulizer determine a duration of a vibration period during a patient or user inhalation to prevent waste of the liquid formulation in an un-inhaled plume of particles.

Additional details regarding the operation of an active mesh nebulizer to perform the method are provided below after the description of the method.

In operation 106 of method 100, a patient is evaluated for symptoms of nicotine intolerance. Symptoms of nicotine intolerance include, nausea, vomiting, perspiration, elevated heart rate, heart palpitations, breathing difficulty, and so forth. Nicotine intolerance is evaluated by providing a patient a first dose (an intolerance evaluation dose) of nicotine at the level or dose determined in operation 104, as described above. A dose of nicotine is delivered to a patient according to the methodology described in U.S. patent application Ser. No. 16/547,072 METHOD OF DELIVERING PHARMACEUTICAL PRODUCTS, filed on Aug. 21, 2019, incorporated herein by reference, using a device as described in U.S. patent application Ser. No. 16/836,485 NEBULIZER WITH CONTROLLED DOSAGE, filed on Mar. 31, 2020.

In some embodiments, nicotine intolerance is evaluated by providing multiple nicotine intolerance evaluation doses, until a cumulative dose results in intolerance symptoms. According to some embodiments, an intolerance evaluation dose of nicotine is delivered in a single breath. In some embodiments, the intolerance evaluation dose is at the lower end of the range of dose sizes described above for operation 104.

In operation 108, based on a result of operation 106 (patient nicotine intolerance evaluation), a determination is made whether to adjust the dose of nicotine for the patient to take to initiate or regulate weight loss for the weight regulation plan. In adjusting the dose of nicotine, a second dose of nicotine is substituted for the first dose in the weight regulation plan. In some embodiments, upon identifying a nicotine dose which results in intolerance symptoms, the nicotine dose is scaled or reduced in order to avoid intolerance while performing the nicotine treatment in a weight regulation plan, and the method continues with operation 110. In some embodiments, the cumulative dose of nicotine which produced nicotine intolerance symptoms is reduced or scaled, by multiplying by a scaling factor ranging from about 0.5 to about 0.8, to achieve a maintaining dose or repeated dose of nicotine to be delivered to the patient.

In some embodiments, a determination is made to keep the nicotine dose unchanged for a patient not exhibiting nicotine intolerance to initiate the weight regulation plan, and the method continues with operation 110.

In operation 110 of method 100, the nicotine dose (the repeated dose, or the maintaining dose) which was determined to not induce nicotine intolerance is delivered to the patient with an active mesh nebulizer. According to embodiments, an entirety of the droplets produced by the active mesh nebulizer are inhaled in order to consume the dose of nicotine. In some embodiments, a single inhalation suffices to consume the dose of nicotine. In some embodiments, multiple inhalations are used to consume the dose of nicotine.

As described above, the dose of nicotine is delivered to a patient with an active mesh nebulizer in order to produce droplets of nicotine solution which have diameters within the range of 0.5 μm to 5 μm. The droplets produced by the active mesh nebulizer are absorbed into the bloodstream through the alveoli in the lungs. The more droplets in the plume of particles which are sized to fit into the alveoli (e.g., having diameters within the range of 0.5 μm to 5 μm), the greater the absorption efficiency of the plume of particles by the lungs. In some embodiments, the percentage of particles having diameters within the range of 0.5 μm to 5 μm is between about 80% and about 85%. In some embodiments, the percentage of particles having diameters within the range of 0.5 μm to 5 μm is between about 85% and about 90%. In some embodiments, the percentage of particles having diameters within the range of 0.5 μm to 5 μm is between about 90% and about 95%. In some embodiments, the percentage of particles having diameters within the range of 0.5 μm to 5 μm is between about 95% and about 99.5%.

According to some embodiments, a dose ranges from about 0.0001 mg nicotine to about 0.0005 mg nicotine. In some embodiments, a dose of nicotine ranges from about 0.0005 mg nicotine to about 0.001 mg nicotine. According to some embodiments, a dose of nicotine ranges from about 0.001 to about 0.002 mg nicotine. In some embodiments, a dose of nicotine ranges from about 0.002 to about 0.005 mg nicotine. According to some embodiments, a dose of nicotine ranges from about 0.005 mg nicotine to about 0.01 mg nicotine. According to theory and belief, doses of nicotine smaller than about 0.0001 mg nicotine are insufficient to promote the metabolic change associated with nicotine-induced weight loss. According to theory and belief, doses of nicotine larger than about 0.01 mg nicotine, when delivered by an active mesh nebulizer, are sufficiently large to induce lethargy in a patient and make normal activities difficult to perform (e.g., driving, operating machinery, and so forth).

Nicotine treatment is configured to deliver a nicotine dose to the patient at a regular interval during the weight regulation plan. A regular interval for nicotine treatment during weight regulation is an interval which induces and maintains a brain effect and metabolic effect which modified a patient metabolism and triggers appetite suppression, and which avoids nicotine cravings in a patient. In general, nicotine consumed by a patient remains in the brain to promote metabolic rate increase and provide satiety, for about 2-3 hours. Thus, according to some embodiments, a regular interval for delivering nicotine during the nicotine treatment an interval of about 2-3 hours. In some embodiments the regular interval is shorter or longer than about 2-3 hours according to individual patient metabolism and nicotine tolerance.

In some embodiments, the active mesh nebulizer is configured to deliver the dose of nicotine to the patient, and then to lock out (or prevent) functioning of the active mesh in order to prevent overdosing by the patient after delivery of the dose of nicotine. In some embodiments, the active mesh nebulizer unlocks functioning of the active mesh upon expiration of a dose delay period (e.g., the regular interval, described above), when a subsequent dose of nicotine is to be provided according to the nicotine treatment in the weight regulation plan. In some embodiments, the dose delay period is about 24 hours. In some embodiments, the dose delay period is between about 3 hours and about 12 hours. In some embodiments, the dose delay period is about 1 hour. In some embodiments, the dose delay period is customized according to a patient's individual response to nicotine metabolism and nicotine craving symptoms.

In operation 112 of method 100, as part of the weight regulation plan, the patient weight is measured after a weight monitoring period (e.g., weekly, or every two weeks, or monthly, or quarterly, and so forth), to determine whether the nicotine dose delivered to the patient according to operation 110 has triggered a metabolic shift/weight loss. In a preferred embodiment of operation 112, a patient weight is monitored every 2 weeks, for 1-2 months, then monitored monthly. A weight monitoring period is adjusted according to a patient convenience, or in response to a reported weight change of the patient. In some embodiments, the weight change is an increase in weight. In some embodiments, the weight change is a decrease in weight. During operation 112 of the method, some patients are expected to experience a weight “plateau” as described above. For patients who experience a weight plateau, the patient and health-care provider discuss weight loss patterns and weight plateaus before determining whether to adjust nicotine dosage.

In operation 114 of method 100, the patient weight is evaluated. In some embodiments, evaluating the patient weight includes comparing the current weight measurement to the weight measured in operation 102, at the start of the weight regulation plan. In some embodiments, evaluating patient weight includes comparing the current weight measurement to a prior weight after nicotine treatment has begun (e.g., after repeating performing operation 110).

In operation 116 of method 100, a determination is made regarding whether patient behavior indicates changing the nicotine dose is indicated. Factors or aspects of patient behavior which can indicate when changing a nicotine dose is indicated include starting exercise, stopping exercise, dietary changes, patient injury, onset of pregnancy, and so forth.

When the evaluation of patient weight (see operation 114) and determining whether patient behavior indicates changing the nicotine dose (operation 116), the operation proceeds to operation 104 to determine a new dose of nicotine.

When the evaluation of patient weight (see operation 114) and determining whether patient behavior does not indicate changing the nicotine dose (operation 116), the operation proceeds to operation 118 and the patient continues to determine a new dose of nicotine.

In some embodiments, a dose of nicotine taken by a patient remains constant throughout a weight management plan. In some embodiments, the dose of nicotine taken by a patient changes throughout a weight management plan. In some embodiments, the nicotine dose increases as the patient initiates weight loss, and decreases once a predetermined amount of weight has been lost. In some embodiments, the nicotine dose remains constant until a patient has experienced two or more consecutive weight measurements with the same weight, at which time the nicotine dose is changed (increased or decreased).

In operation 118 of method 100, the dose of nicotine is delivered to the patient as described above in operation 110 until a weight monitoring period has elapsed, and the method 100 continues with operation 112.

Examples of a nicotine treatment are provided to clarify the dosing protocol described above, and to clarify the effect of a nicotine dose on a patient.

Example 1: A patient is treated with nicotine solution from an active mesh nebulizer having a nebulization rate of 0.2 milliliters of solution per minute (0.2 ml/min). The nicotine solution used for nicotine treatment contains 0.05 milligram of nicotine (free base) per milliliter of solution (0.1 mg/ml). The active mesh nebulizer activates the active mesh for a single inhalation period of 3 seconds (0.05 minute) to deliver the dose of nicotine to the patient. Thus, the nicotine dose delivered to the patient is determined to be:

(0.2 ml/min.)(0.05 mg/ml)(0.05 min.)=0.0005 mg. Equation (1)

The male patient is a 2-pack per day smoker and finds that the dose calculated by Equation (1) delivered approximately every two hours is sufficient to manage nicotine cravings while holding the patient's weight at the same level as when the patient was smoking cigarettes.

Example 2: A patient is treated with nicotine solution from an active mesh nebulizer having a nebulization rate of 0.2 milliliters of solution per minute (0.2 ml/min). The nicotine solution used for nicotine treatment contains 0.5 milligram of nicotine (free base) per milliliter of solution (0.5 mg/ml). The active mesh nebulizer activates the active mesh for a single inhalation period of 3 seconds (0.05 minute) to deliver the dose of nicotine to the patient. Thus, the nicotine dose delivered to the patient is determined to be:

(0.2 ml/min.)(0.5 mg/ml)(0.05 min.)=0.005 mg. Equation (2)

The male patient is a 2-pack per day smoker and finds that the dose calculated by Equation (2) is sufficiently large to produce lethargy, or lassitude, making normal daily functions difficult to perform.

Example 3: A hypothetical patient is treated with nicotine solution from an active mesh nebulizer having a nebulization rate of 0.2 milliliters of solution per minute (0.2 ml/min). The nicotine solution used for nicotine treatment contains 0.01 milligram of nicotine (free base) per milliliter of solution (0.01 mg/ml). The active mesh nebulizer activates the active mesh for a single inhalation period of 3 seconds (0.05 minute) to deliver the dose of nicotine to the patient. Thus, the nicotine dose delivered to the patient is determined to be:

Equation (3)

The hypothetical patient receives the nicotine dose at a regular interval (e.g., about every 2 hours) for a weight monitoring period (e.g., about 2 months in the present hypothetical example), after which the hypothetical patient is weighed to determine the effect of the nebulized nicotine solution on the hypothetical patient's weight. On determining that the hypothetical patient has lost weight, the patient treatment continues with the same nicotine dose until the patient's weight loss has stabilized for two consecutive weight monitoring periods (e.g., about 4 months in the present hypothetical example), at which time a determination is made about whether to increase the nicotine dose and attempt further weight loss, or to leave the nicotine dose unchanged and maintain the patient's new lower weight.

According to theory and belief, the small particle size and increased delivery efficiency of droplets of the active mesh nebulizer nicotine source solution produces a brain response in less than 10 seconds, comparable to or faster than the brain response from smoking tobacco or using an electronic cigarette. The rapid brain response experienced by patients using an active mesh nebulizer to ingest nicotine solutions is believed to be associated with greater patient adherence to a weight regulation treatment plan.

According to theory and belief, a nicotine solution having 0.5 mg nicotine/ml of solution (e.g., 0.05%, whether free base or dissolve nicotine salt) delivered to a nicotine user with a single inhalation plume from an active mesh nebulizer with a nebulization rate of 0.2 ml/minute for a period of 10 seconds produces a strong brain effect, including profound relaxation, with no desire for further nicotine inhalation for between 2-3 hours. According to theory and belief, a nicotine solution having 1/40 mg nicotine/ml solution (e.g., 0.025% nicotine) is also able to produce nicotine satiety for a period of at least two hours (nebulization rate of 0.2 ml/min nebulization rate, nebulization time of 10 seconds) also produced a strong brain effect, with no desire for further nicotine inhalation for at least two hours.

Additional operational details regarding the performance of the method described herein with an active mesh nebulizer are provided below.

In some embodiments, based on the instructions, the active mesh nebulizer determines a duration of a total vibration period, to deliver a full dose of medication to a patient or user. A full dose of medication is a quantity prescribed for periodic delivery to the patient or user by the medical provider. In some embodiments, according to a concentration of medication (a single compound, or a mixture of multiple compounds) in the liquid formulation, the total vibration period to deliver the full dose of medication to the user is shorter than a duration of a single patient inhalation. The total vibration period to deliver a full dose of medication is based on the characterized nebulization rate of the active mesh, the viscosity of the liquid formulation, the quantity of liquid in contact with the mesh (e.g., the coverage area of the liquid on the active mesh) during mesh vibration, the patient or user lung volume, the lung inflation rate for the patient or user, the absorptive surface area of the lung (which may be compromised by medical conditions such as emphysema), and so forth. During operation of an active mesh nebulizer, nebulization rate is a function of at least the active mesh vibration rate, the diameter and number of holes in the mesh, and the voltage applied to the piezoelectric vibrating element. Droplet formation by the active mesh is a function of the liquid formulation viscosity. In some embodiments, a mesh produces an acceptable plume of particles for inhalation into the lungs for a range of liquid formulation viscosities, and a different active mesh is indicated to produce plumes of particles of liquid formulations having a viscosity outside the range of the initial active mesh performance specification. Mesh coverage area also relates to the rate of plume generation. When an entirety of the active mesh is in contact with a liquid formulation, the nebulization/plume generation rate is greater than during operation of the active mesh having only half of the active mesh in contact with the liquid formulation. Plume generation efficiency, and dosing accuracy, is improved by an active mesh nebulizer configured to promote greater amounts of mesh coverage by the liquid formulation.

In some embodiments, the instructions include a programmed total dose of medication for a patient or user, and the total vibration period is determined by dividing the total dose by the nebulization rate of the active mesh nebulizer. In embodiments of the method where the total vibration period is longer than the inhalation period of which a patient or user is capable (due to, e.g., physiological constraints), or expected of a patient or user (due to, e.g., age of the user, or mental capacity), the total vibration period is divided into smaller sub-dose vibration periods and the total dose of medication is provided to the patient or user over multiple inhalations. In embodiments of the method where the total vibration period is less than the inhalation period of which a patient or user is capable (due to, e.g., physiological constraints), or expected of a patient or user (due to, e.g., age of the user, or mental capacity), the total dose of medication is provided to the patient or user in a single inhalation period.

In some embodiments, the patient is alerted to begin inhalation in order to receive a total dose, or a sub-dose, of medication. In some embodiments, the patient alert comprises a vibration of the active mesh nebulizer while the patient or user holds the nebulizer against the patient or user's mouth for inhalation. In some embodiments, the patient alert comprises a sound or tone generated to alert the user to begin inhalation. In some embodiments, the patient alert comprises a visual alert (e.g., a blinking light or visual indicator) to begin inhalation. In some embodiments, the patient alert is provided by the active mesh nebulizer. In some embodiments, the patient alert is provided by a computing device communicatively connected to the active mesh nebulizer to facilitate patient treatment and/or active mesh nebulizer operation. Examples of a computing device communicatively connected to the active mesh nebulizer include a dedicated nebulizer controller unit, a “smartphone,” a “feature-rich” phone, a computing tablet, or any other kind of computing device configured with software instructions and a communication channel to communicate with the active mesh nebulizer and interact with the patient or user. In some embodiments, the patient alert comprises at least two of more of a sound, a vibration (tactile alert), or a visual alert of the active mesh nebulizer and/or the connected computing device.

According to theory and belief, a patient inhalation typically ranges from about three (3) seconds to about ten (10) seconds before a patient has inhaled sufficient air to inflate the lungs to a maximum lung volume. In some embodiments, inhalation may occur over timespans ranging from 10 seconds to 20 seconds, and the slower rate of inhalation is believed to have an impact on the distribution of inhaled particles in the lungs, and on the absorption rate of the medication. In some embodiments, the amount of time a patient or user is able to inhale is influenced by lung volume, bronchial diameter, and so forth.

In some embodiments, patient age or mental capacity of a patient is a factor in the amount of time a patient or user is able to inhale, and/or the spacing between inhalation periods. For young patients, or patients with cognitive impairment, spacing between inhalation periods to deliver a dose of medication is longer than for adults or patients with no cognitive impairment in order to provide the patient an opportunity to prepare for a possible second inhalation period for a multi-inhalation medication delivery scenario. According to some embodiments, the instructions are configured to scale the duration of the inhalation period based on (e.g., approximately proportional to) the patient lung volume, as compared to a patient having no physiological impairment. In some embodiments, instructions to the active mesh nebulizer for determining a total number of inhalation periods of an active mesh nebulizer are adjusted according to the age and gender of the patient or user, the measured lung volume of the patient, the peak flow (during exhalation) measurement of a patient or user, or other physiological factors such as surgical history (e.g., whether portions of the lungs have been removed), heart volume, patient weight, fluid buildup around the heart or lungs, and the like.

In some instances, the instructions include a programmable vibration start delay period between the start of an alert to a patient to begin inhalation, an end of the patient alert, or some other factor associated with timing of inhaling the plume of particles. According to some embodiments, the duration of a programmable vibration start delay period ranges from about 1 second to about 3 seconds. Vibration start delay periods shorter than about 1 second are believed more likely to result in wasted plume than for longer delay periods. Vibration start delay periods longer than about 3 seconds are believed to be, for most patients, of such length that the patient has begun inhaling before the plume is generated, inflating the lungs with air that does not contain particles of liquid formulation of the medication, making inhalation of the whole plume of particles more difficult. While some patients are expected to find such vibration start delay periods unacceptable (e.g, uncomfortable or inconvenient), the net result is to merely increase a number of inhalation periods to deliver a full dose of medication. For patients with cognitive impairment, or young patients, a vibration delay period of about 3 seconds, or longer, is appropriate to accommodate the different response time or concentration the patient or user is able to pay to the treatment process. The vibration start delay period is intended to be programmable by a medical provider or other person monitoring performance of the patient or user such that the patient or user becomes habituated to a customized treatment process adapted to the patient or user's individual ability. Successful adaptation of the patient or user creates an opportunity for patient self-medication for some medications, without constant oversight by a medical provider.

The process of generating the plume of particles is halted before the patient stops the inhalation in order to avoid wasting the medication in the un-inhaled ending portion of the plume of particles. According to some embodiments, the patient is signaled as to the duration of the inhalation period by an alert from the active mesh nebulizer, or a connected computing device. In some embodiments, the patient is provided a periodic alert to track or count to indicate continuing plume generation and avoid inadvertent wasting of a last portion of the plume of particles. In some embodiments, the patient is provided an “ending” alert, different from a starting alert, and/or different from a “tracking” or “counting” alert, to indicate that the plume generation has halted. In some embodiments, a patient is provided an alert (a “further inhalation” alert) to indicate that further inhalation periods are upcoming to prepare for additional plume generation. In some embodiments, a patient is provided an alert (a “final” alert) to indicate that the total dose of medication has been delivered and no further inhalations are upcoming until another full dose of medication is scheduled. In some embodiments, the alerts are tones, vibrations, visual alerts, or recorded messages indicating a status of the active mesh nebulizer or a treatment status (a number of sub-doses delivered, a number of sub-doses remaining, an anticipated number of doses remaining in the vial, and so forth). “Coordinating” alerts to guide a patient or user to start, maintain, or halt inhalation around the programmed generation of the plume of particles are customizable according to the patient's ability to process and adhere to a treatment protocol, and are used singly, or in combination, in order to achieve patient compliance with treatment.

In some embodiments, the active mesh nebulizer controller determines whether the dose of medication has been delivered according to the instructions received by the active mesh nebulizer. Upon determining that the total dose of medication has not been delivered, the method repeats the plume generation after a short delay. Upon determining that the total dose of medication has been delivered, the active mesh nebulizer performs a lockout function to prevent accidental overdosing. In some embodiments, the active mesh nebulizer operates under a “fixed dosage” model and is halted until a full dose delay period expires, when an additional full dose of medication is delivered from the active mesh nebulizer in one or more inhalations. In “fixed dosage” operation, the halt between treatment by the active mesh nebulizer is complete and no intermediate operation is allowed (see, e.g., partial dosing, or “running” dosing, below). In some embodiments, the active mesh nebulizer operates under a “partial dosage” model and, upon completion of a full dose of medication, is completely halted until a partial dose delay period expires, at which time a the active mesh nebulizer activates to delivery of a partial dose of medication, but not a full dose of medication. In “partial dosage” operation, the nebulizer becomes available for a treatment with a partial dose of medication from the expiration of the partial delay period until the expiration of the full dose delay period, at which point the nebulizer becomes available for treatment with a full dose of medication.

In some embodiments, the active mesh nebulizer operates a “running dosage” model, wherein the active mesh nebulizer remains available for additional treatment with the medication at all times, unless an additional partial or full treatment of medication will exceed a blood serum concentration, or some other physiological threshold, of the medication in the patient or user. As the amount of delivered medication approaches a calculated blood serum concentration (threshold, especially for tissue toxicity or other adverse effect), the nebulizer completely halts operation until such time as the calculated blood serum concentration, or other physiological threshold, will not be exceeded by an additional partial or full dose of medication. A mode of delivery of medications from the active mesh nebulizer is a function of the chemical nature of the medication, the patient's metabolic ability with regard to the medication, and the patient's sensitivity to the medication, among others. The instructions of the nebulizer are configured to account for the aforementioned factors to avoid over dosage or other adverse patient outcomes based on taking the medication.

The particles produced by a suitable active mesh nebulizer have a particle diameter ranging from about 0.5 micrometers (μm or microns) to about 5 micrometers. Particles are carried into the lungs during inhalation. Because of the small initial diameters, the produced particles entrained by the inhaled air and carried deep into the lungs, making little or no contact with the walls of the mouth, the throat, and the upper portions of the lungs. It is believed that the small particles absorb some water during inhalation. It is further believed that, despite any water absorption during inhalation, the particles are sufficiently small to undergo efficient entry into the alveoli. In the alveoli, particles contact the alveolar walls and are absorbed into the flow of freshly-oxygenated blood returning from the lungs to the heart. On exiting the heart, oxygenated blood divides into two portions: a first portion of about 20% traveling to the brain, and a second portion of about 80% traveling to a remainder of the body.

Aspects of the present disclosure are associated with a method which includes steps of measuring a patient weight prior to delivering a dose of nicotine to the patient; and delivering the dose of nicotine to the patient by inhalation from an active mesh nebulizer at a regular interval, wherein the dose of nicotine is not larger than 0.01 mg and not smaller than 0.0001 mg. In some embodiments, delivering a dose of nicotine to a patient by inhalation from an active mesh nebulizer at a regular interval further includes: generating a plume of particles of a nicotine solution in the active mesh nebulizer after the start of a patient inhalation; and stopping generating the plume of particles of the nicotine solution before the end of the patient inhalation. In some embodiments, the method further includes steps for calculating, based on an active mesh nebulizer nebulization rate, a nicotine concentration in the nicotine solution, and the dose of nicotine, a number of times the active mesh nebulizer activates the active mesh to generate the plume of particles. In some embodiments, the method includes evaluating the patient for symptoms of nicotine intolerance subsequent to delivering the dose of nicotine to the patient by inhalation from the active mesh nebulizer. In some embodiments, the methods include determining the dose of nicotine by delivering at least one incremental dose of nicotine from the active mesh nebulizer until the patient exhibits symptoms of nicotine intolerance. In some embodiments, the dose is the at least one incremental dose of nicotine multiplied by a scaling factor ranging from 0.5 to 0.8. In some embodiments, the method further includes determining whether to change the dose of nicotine delivered by inhalation from the active mesh nebulizer. In some embodiments, determining whether to change the dose of nicotine to the patient further includes determining whether the patient has performed dietary changes; and determining whether the patient has modified exercise or physical activity levels. In some embodiments, the method includes decreasing the dose of nicotine upon determining that the patient has performed dietary changes and/or modified exercise or physical activity levels. In some embodiments, the method includes increasing the dose of nicotine upon determining that the patient has not performed dietary changes and/or modified exercise or physical activity levels. In some embodiments, the method includes repeating determining the dose of nicotine by delivering the at least one incremental dose of nicotine from the active mesh nebulizer until the patient exhibits symptoms of nicotine intolerance subsequent to delivering the dose of nicotine to the patient by inhalation from an active mesh nebulizer at a regular interval.

Aspects of the present disclosure relate to a method which includes steps of generating a plume of droplets of a nicotine solution with an active mesh nebulizer; causing a patient to inhale the entire plume of droplets into the lungs; evaluating the patient for nicotine intolerance symptoms; and repeating generating the plume of droplets and causing the patient to inhale the entire plume of droplets at a regular interval upon determining that the patient does not exhibit nicotine intolerance. In some embodiments, the method includes steps of measuring a patient weight; and repeating monitoring the patient weight after a first weight monitoring period, wherein the weight monitoring period is a period of at least one week. In some embodiments, the plume of droplets of the nicotine solution includes a first nicotine dose, and further includes substituting a second nicotine dose for the first nicotine dose in the plume of droplets of the nicotine solution. In some embodiments, repeating generating the plume of droplets and causing the patient to inhale the entire plume of droplets at a regular interval further includes generating the plume of droplets including the second nicotine dose. In some embodiments, the method further includes steps for evaluating the patient for nicotine intolerance symptoms after: substituting the second nicotine dose for the first nicotine dose, a first instance of generating the plume of droplets having the second nicotine dose, and a first instance causing the patient to inhale the entire plume of droplets having the second nicotine dose. In some embodiments, substituting a second nicotine dose for the first nicotine dose in the plume of droplets of the nicotine solution is performed upon determining that the patient weight is unchanged after at least two weight monitoring periods. In some embodiments, substituting a second nicotine dose for the first nicotine dose in the plume of droplets of the nicotine solution includes substituting a larger dose for a smaller dose. In some embodiments, substituting a second nicotine dose for the first nicotine dose in the plume of droplets of the nicotine solution includes substituting a smaller dose for a larger dose.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method, comprising:
measuring a patient weight prior to delivering a dose of nicotine to the patient; and
delivering the dose of nicotine to the patient by inhalation from an active mesh nebulizer at a regular interval, wherein the dose of nicotine is not larger than 0.01 mg and not smaller than 0.0001 mg.

2. The method of claim 1, wherein delivering a dose of nicotine to a patient by inhalation from an active mesh nebulizer at a regular interval further comprises:
generating a plume of particles of a nicotine solution in the active mesh nebulizer after the start of a patient inhalation; and
stopping generating the plume of particles of the nicotine solution before the end of the patient inhalation.

3. The method of claim 2, further comprising:
calculating, based on an active mesh nebulizer nebulization rate, a nicotine concentration in the nicotine solution, and the dose of nicotine, a number of times the active mesh nebulizer activates the active mesh to generate the plume of particles.

4. The method of claim 1, further comprising:
evaluating the patient for symptoms of nicotine intolerance subsequent to delivering the dose of nicotine to the patient by inhalation from the active mesh nebulizer.

5. The method of claim 4, further comprising:
determining the dose of nicotine by delivering at least one incremental dose of nicotine from the active mesh nebulizer until the patient exhibits symptoms of nicotine intolerance.

6. The method of claim 5, wherein the dose is the at least one incremental dose of nicotine multiplied by a scaling factor ranging from 0.5 to 0.8.

7. The method of claim 1, further comprising determining whether to change the dose of nicotine delivered by inhalation from the active mesh nebulizer.

8. The method of claim 7, wherein determining whether to change the dose of nicotine to the patient further comprises:
determining whether the patient has performed dietary changes; and
determining whether the patient has modified exercise or physical activity levels.

9. The method of claim 8, further comprising decreasing the dose of nicotine upon determining that the patient has performed dietary changes and/or modified exercise or physical activity levels.

10. The method of claim 8, further comprising increasing the dose of nicotine upon determining that the patient has not performed dietary changes and/or modified exercise or physical activity levels.

11. The method of claim 10, further comprising repeating determining the dose of nicotine by delivering the at least one incremental dose of nicotine from the active mesh nebulizer until the patient exhibits symptoms of nicotine intolerance subsequent to delivering the dose of nicotine to the patient by inhalation from an active mesh nebulizer at a regular interval.

12. A method, comprising:
generating a plume of droplets of a nicotine solution with an active mesh nebulizer;
causing a patient to inhale the entire plume of droplets into the lungs;
evaluating the patient for nicotine intolerance symptoms; and
repeating generating the plume of droplets and causing the patient to inhale the entire plume of droplets at a regular interval upon determining that the patient does not exhibit nicotine intolerance.

13. The method of claim 12, further comprising;
measuring a patient weight; and
repeating monitoring the patient weight after a first weight monitoring period, wherein the weight monitoring period is a period of not less than one week.

14. The method of claim 13, wherein the plume of droplets of the nicotine solution comprises a first nicotine dose, and further comprising substituting a second nicotine dose for the first nicotine dose in the plume of droplets of the nicotine solution.

15. The method of claim 14, wherein repeating generating the plume of droplets and causing the patient to inhale the entire plume of droplets at a regular interval further comprises generating the plume of droplets comprising the second nicotine dose.

16. The method of claim 14, further comprising evaluating the patient for nicotine intolerance symptoms after:
substituting the second nicotine dose for the first nicotine dose, a first instance of generating the plume of droplets having the second nicotine dose, and
a first instance causing the patient to inhale the entire plume of droplets having the second nicotine dose.

17. The method of claim 14, wherein substituting a second nicotine dose for the first nicotine dose in the plume of droplets of the nicotine solution is performed upon determining that the patient weight is unchanged after at least two weight monitoring periods.

18. The method of claim 14, wherein substituting a second nicotine dose for the first nicotine dose in the plume of droplets of the nicotine solution comprises substituting a larger dose for a smaller dose.

19. The method of claim 14, wherein substituting a second nicotine dose for the first nicotine dose in the plume of droplets of the nicotine solution comprises substituting a smaller dose for a larger dose.

20. A method, comprising:
measuring a patient weight;
determining a first dose of nicotine by scaling a cumulative dose of nicotine;
delivering the first dose of nicotine to a patient by inhalation from an active mesh nebulizer at a repeated interval;
measuring the patient weight after delivering the first dose of nicotine to a patient by inhalation from an active mesh nebulizer at a repeated interval; and
determining whether to replace the first dose of nicotine with a second dose of nicotine different from the first dose of nicotine; and
delivering the second dose of nicotine to a patient by inhalation from the active mesh nebulizer at the repeated interval.
